(19) United States
(12) Patent Application Publication
(10) Pub. No.: US 2008/0114454 A1
(43) Pub. Date: May 15, 2008

(54) Expandable Vertebral Body Implants Including Shape-Memory Materials and Methods of Use

(75) Inventors: Marc Peterman, Austin, TX (US); Lindsey Gardner Waugh, Memphis, TN (US); Jason A. Edie, Memphis, TN (US)

Correspondence Address: COATS & BENNETT/MEDTRONICS, 1400 CRESCENT GREEN, SUITE 300, CARY, NC, 27518, US

(73) Assignee: Warsaw Orthopedic, Inc., Warsaw, IN (US)

(21) Appl. No.: 11/558,490

(22) Filed: Nov. 10, 2006

(51) Int. Cl. A61F 2/44

(52) U.S. Cl. 623/17.16

(57) ABSTRACT

A vertebral implant for insertion into a patient includes a tubular member extending along a longitudinal axis and includes a first region comprised of a shape-memory material. The first region includes a first longitudinal height when the implant is maintained at a temperature below a threshold temperature. The first region includes a second longitudinal height when the implant is maintained at a temperature at or above the threshold temperature. The tubular member further includes a second region with a third longitudinal height regardless of whether the implant is maintained above or below the threshold temperature. The second region may be constructed of the same shape-memory material. The second region may be disposed at an end of the tubular member. Bone-growth materials may be packed into the ends of the tubular member. End pieces may be coupled to the ends of the tubular member.

### BACKGROUND

Spinal implants are often used in the surgical treatment of spinal disorders such as degenerative disc disease, disc herniations, scoliosis or other curvature abnormalities, and fractures. Many different types of treatments are used, including the removal of one or more vertebral bodies and/or intervertebral disc tissue. In some cases, spinal fusion is indicated to inhibit relative motion between vertebral bodies. In other cases, dynamic implants are used to preserve motion between vertebral bodies. In yet other cases, relatively static implants that exhibit some degree of flexibility may be inserted between vertebral bodies.

Regardless of the type of treatment and the type of implant used, surgical implantation tends to be a difficult for several reasons. For instance, access to the affected area may be limited by other anatomy. Further, a surgeon must be mindful of the spinal cord and neighboring nerve system. The size of the implant may present an additional obstacle. In some cases, an implant with a desired height may be difficult to insert. The implant may interfere with distraction tools or the patient's anatomy. Expandable implants are becoming more prevalent as a response to some of these concerns. However, the expansion mechanism in these devices tends to be complex or large. Consequently, existing devices do not appear to address each of these issues in a manner that improves the ease with which the device may be surgically implanted.

### SUMMARY

Illustrative embodiments disclosed herein are directed to a vertebral implant for insertion between vertebral bodies in a patient. The implant includes a tubular member extending along a longitudinal axis and includes a first region comprised of a shape-memory material. The first region includes a first longitudinal height when the implant is maintained at a temperature below a threshold temperature. The first region includes a second longitudinal height when the implant is maintained at a temperature at or above the threshold temperature. The tubular member further includes a second region with a third longitudinal height regardless of whether the implant is maintained above or below the threshold temperature. The second region may be constructed of the same shape-memory material. The second region may be disposed at an end of the tubular member. Thus, bone-growth materials may be packed into the ends of the tubular member and should not become dislodged when the first region expands. End pieces may be coupled to the ends of the tubular member.

The implant may be chilled to hold a compressed state. The first region may be shaped and/or sized to deform before the second region when the implant is chilled. Further, deforming the implant into the compressed state may include laterally constraining the implant to prevent radial deformation. Accordingly, when the implant expands, it may expand in the longitudinal direction.

### BRIEF DESCRIPTION OF THE DRAWINGS

FIG. 1 is a lateral view of a vertebral implant according to one or more embodiments shown relative to vertebral bodies;

FIG. 2 is a lateral view of a vertebral implant in a compressed state according to one or more embodiments;

FIG. 3 is a lateral view of a vertebral implant in an expanded state according to one or more embodiments;

FIG. 4 is an axial view of a vertebral implant according to one or more embodiments;

FIG. 5 is an axial view of a vertebral implant according to one or more embodiments;

FIG. 6 is an axial view of a vertebral implant according to one or more embodiments;

FIG. 7 is a lateral view of a vertebral implant according to one or more embodiments;

FIG. 8 is an axial view of a vertebral implant according to one or more embodiments;

FIG. 9 is a lateral view of a vertebral implant in a compressed state according to one or more embodiments shown relative to vertebral bodies;

FIG. 10 is a lateral view of a vertebral implant in an expanded state according to one or more embodiments shown relative to vertebral bodies;

FIG. 11 is a lateral view of a vertebral implant in a compressed state according to one or more embodiments;

FIG. 12 is a lateral view of a vertebral implant in an expanded state according to one or more embodiments;

FIG. 13 is a lateral view of a vertebral implant in a compressed state according to one or more embodiments;

FIG. 14 is a lateral view of a vertebral implant in an expanded state according to one or more embodiments;

FIG. 15 is a lateral view of a vertebral implant according to one or more embodiments shown constrained against radial displacement during compression; and.

FIG. 16 is a lateral view of a vertebral implant following axial compression according to one or more embodiments.

### DETAILED DESCRIPTION

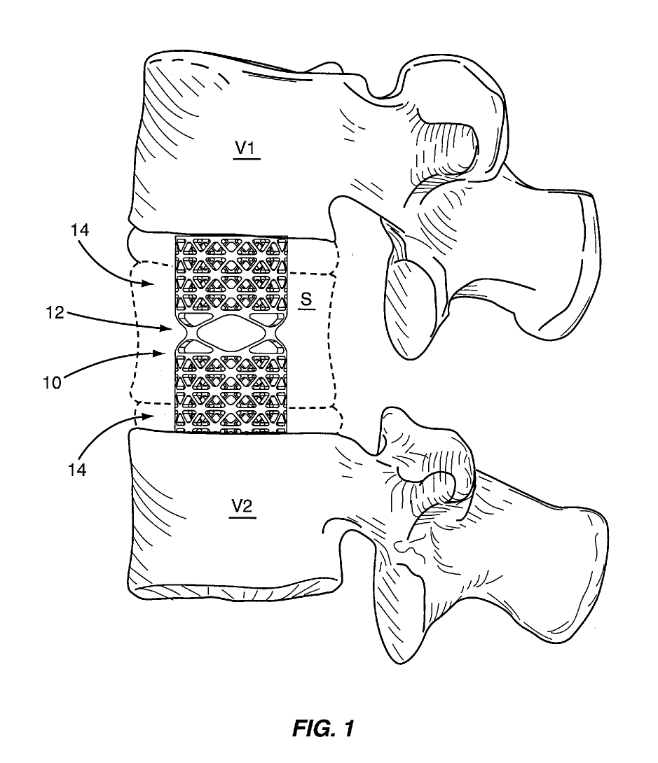

The various embodiments disclosed herein relate to a vertebral implant in which a discrete body may be formed from a shape-memory material that expands upon the introduction of elevated temperatures to establish a desired spacing between vertebral bodies in a patient. Advantageously, the implant may be inserted in a compressed state with the implant expanding to a desired spacing in situ. Reference number 10 in FIG. 1 generally identifies an exemplary implant. In one embodiment, the implant 10 is positionable within an intervertebral space S to span one or more vertebral levels along the longitudinal axis of the spinal column. Although the illustrated embodiment of the implant 10 spans one vertebral level, it should be understood that the implant 10 may be configured to span multiple vertebral levels, including two or more vertebral levels. As described herein, the term “vertebral level” is intended to mean the combination of a vertebral body and one or both adjoining discs. As such, the implant 10 may be implemented in vertebrectomy or corpectomy procedures as are known in the art.

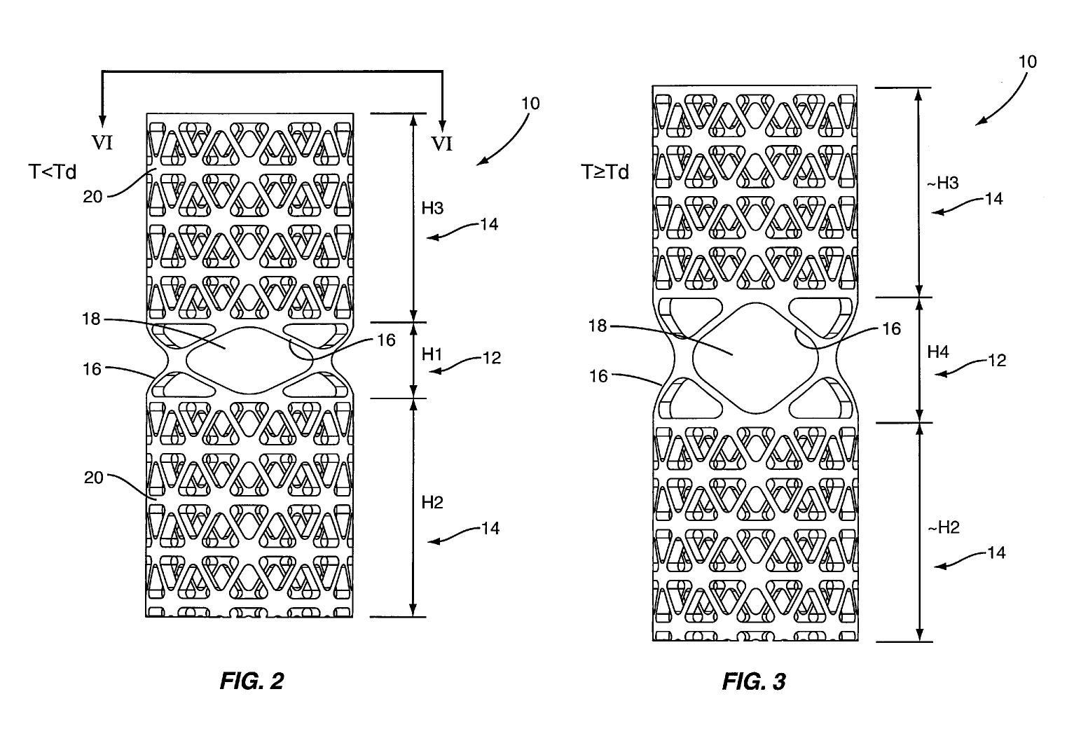

In the illustrated embodiment, the implant 10 generally includes tubular shape that includes an expanding portion 12 and at least one substantially non-expanding portion 14. In the embodiment shown, the expanding portion 12 is disposed near a center of the tubular implant 10 while the separate non-expanding portions 14 are disposed towards the ends of the tubular implant 10. The expanding portion 12 is characterized by a material and a geometry that permits expansion upon exposure to elevated temperatures. Conversely, the non-expanding portions 14 are characterized by a material and/or a geometry that substantially restricts expansion upon exposure to the same elevated temperatures. Generally, the expanding portion 12 may be constructed from a shape-memory material that includes metals or polymers. These types of materials are known in the art. The expanding portion 12 may be compressed and stored at a shortened installation height as shown in FIG. 2 and allowed to expand as shown in FIG. 3 after implantation between vertebral bodies in a patient. Limiting the expanding portion 12 to a particular region of the tubular implant 10 offers several advantages as are described below.

In one embodiment, the expanding portion 12 is fabricated from a shape-memory polymer (SMP) material that can be molded into a desired configuration. Curing the polymeric material imprints the original molded configuration to the spacer body. However, when the spacer body is heated above a deformation temperature (Td)—which is usually equivalent to the glass transition temperature (Tg) of the polymeric material—the SMP becomes elastic. When heated to a temperature equal to or above Td, the spacer body can be deformed to a wide variety of configurations by applying pressure or forcing it into a mold. The spacer body can be “frozen” into the deformed configuration by cooling it below the Td while the body is maintained in the deformed configuration. Thereafter, the deformed spacer body retains the deformed configuration until it is heated above Td. When the spacer body is reheated above Td, the SMP material again becomes elastic; and in the absence of any applied pressure, the spacer body automatically reverts to it original configuration. This process can be repeated any number of times without detrimental effect on the SMP material or the spacer itself.

In another embodiment, the expanding portion 12 may be fabricated from a shape-memory alloy (SMA), such as, for example, the nickel-titanium alloy known as Nitinol. The response of the shape memory material to deformation generally has two triggers as known in the art to induce the material to partially or fully recover its memorized shape. The first trigger is a thermal trigger where the deformed state is initially at a temperature such that the deformed state is stable. Upon heating, the temperature rises until the deformed state is no longer stable and begins to change to the memorized state. Accordingly, the transition temperature Td for the material may be controlled (by varying the alloy composition) so that a chilled (e.g. −5 degrees Celsius) implant 10 expands upon exposure to normal body temperatures around 35-40 degrees Celsius. Once the expanding portion 12 expands upon exposure to the elevated temperatures, the tubular implant 10 remains substantially rigid to provide a stable fusion site.

The second trigger is a stress-actuated trigger. The undeformed state is at a temperature such that at least some of the material is in the austenitic state, where the material behaves similar to Titanium. Under the influence of sufficient stress, the austenitic material will transform into the martensitic state. Upon the release of some or all of the stress, the temperature is such that the martensitic state is unstable and will automatically attempt to revert to the austenitic state with consequent shape reformation. It should also be understood that the shape memory material may attempt to recover the memorized shape by using some combination of thermal and stress actuation. Accordingly, in one embodiment, the transition temperature for the material may be controlled so that upon exposure to normal body temperatures around 35-40 degrees Celsius, the implant 10 exhibits superelastic properties offering some relative motion between vertebral bodies.

The non-expanding portions 14 may be constructed from a biocompatible material, such as, for example, a carbon fiber material, or non-metallic substances, including polymers or copolymers made from materials such as PEEK and UHMWPE. In further embodiments, the non-expanding portions 14 may be formed of other suitable biocompatible materials, such as, for example, stainless steel, titanium, and cobalt-chrome.

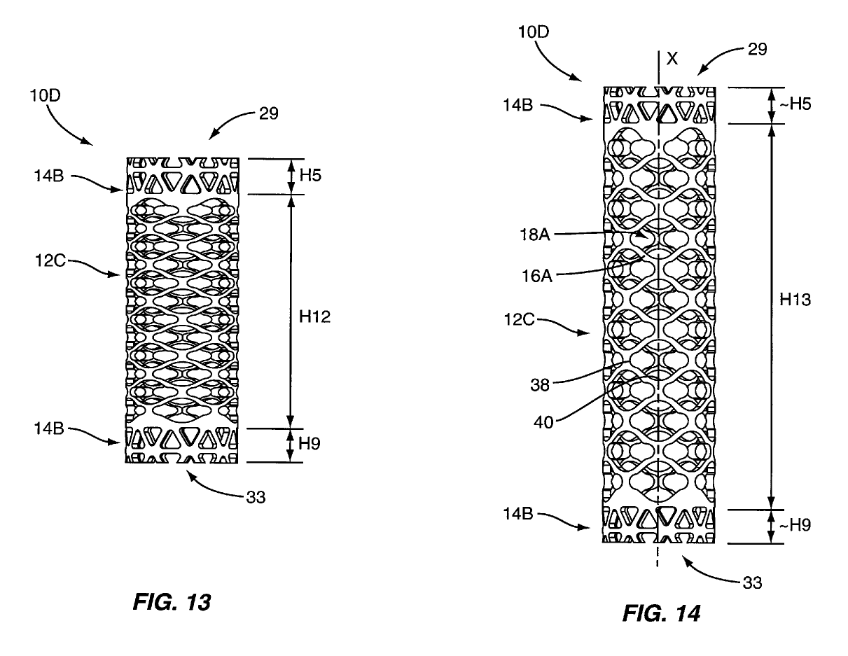

In one embodiment, the non-expanding portions 14 may be constructed from the same shape-memory material as the expanding portion 12. Notably, the non-expanding portions 14 may include a rigid geometry that resists compression, even when the material is chilled or stressed. As FIGS. 2 and 3 show, the expanding portion 12 includes elongated linking segments 16 forming enlarged apertures 18 therein. The enlarged apertures may include a geometric shape, such as the diamond shape illustrated. In other embodiments, the apertures 18 may include other polygonal shapes with rounded or non-rounded corners. In other embodiments, the apertures 18 may include oval or circular shapes. In other embodiments, such as that shown in FIGS. 13 and 14 and described below, may include non-geometric shapes. The non-expanding portions 14 may include a solid outer wall 20 or a perforated configuration as shown. To the extent the non-expanding portions 14 include a perforated geometry, the linking segments 16 should include a lower yield strength than the walls 20 of the non-expanding portion 14. With this characteristic, the expanding portion 12 will yield and the non-expanding portion will not substantially yield during compression of the shape-memory material.

Consequently, when the tubular implant 10 expands upon exposure to elevated temperatures, the expanding portion 12 expands from a first height Hi as shown in FIG. 2 to a second height H4 as shown in FIG. 3. Correspondingly, the more rigid non-expanding portions 14 will remain at or near the same height in both the martensitic state (see dimensions H2, H3 in FIG. 2) and the austenitic state (see dimensions ˜H2, ˜H3 in FIG. 3). As a result, the change in overall height of the implant 10 from FIG. 2 (H1+H2+H3) to the overall height of the implant 10 in FIG. 3 (H4+(˜H2)+(˜H3)) is largely attributable to the change in height H4−H1 of the expanding portion 12. In one embodiment, the change in height H4−H1 of the expanding portion 12 is greater than or equal to about 5% of the overall height of the implant 10. Obviously, larger percentages (20%, 40%, etc. . . . ) are possible and may be desirable for a particular application. In one embodiment, the change in height in the non-expanding portions 14 ((˜H2)+(˜H3)-H2-H3) is less than or equal to about 5% of the overall height of the implant 10.

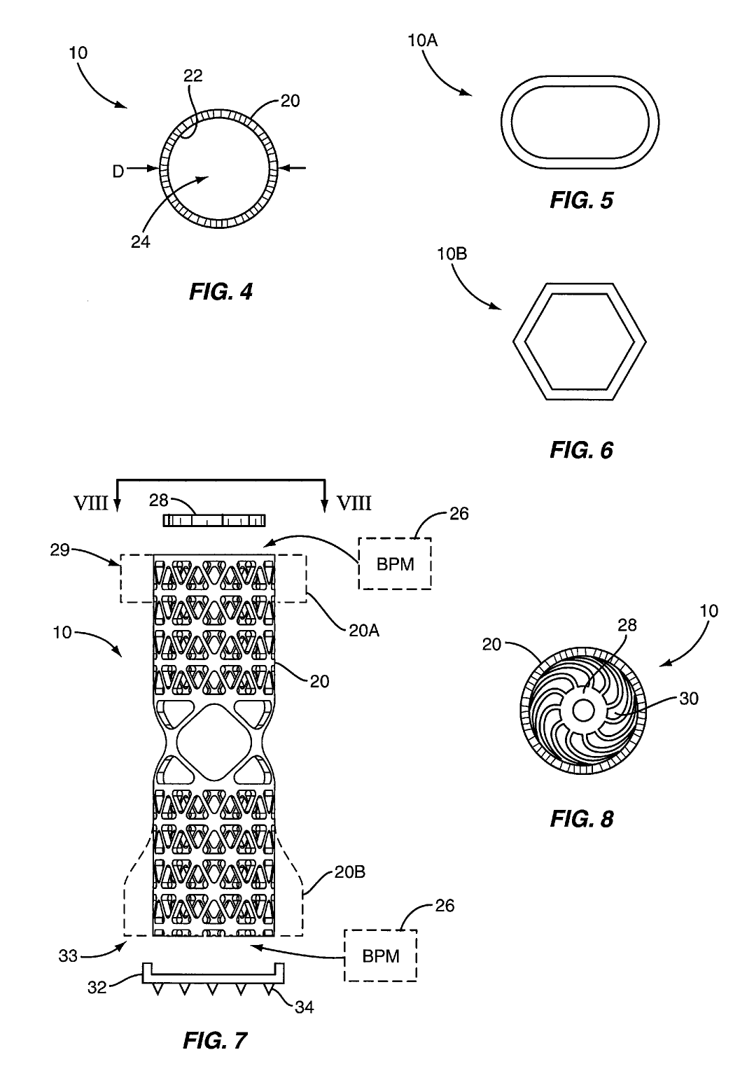

FIG. 4 shows an end view of the exemplary tubular implant 10 according to the view lines provided in FIG. 2. The tubular implant 10 is generally hollow along its length with an interior volume 24 formed by the inner surface 22 of the exterior walls 20. In the embodiment shown, the tubular implant 10 includes a substantially circular cross section defined by an outer diameter D. In other embodiments, the tubular implant 10 may include different cross section shapes. For example, FIG. 5 shows an tubular implant 10A including an asymmetric cross section. The asymmetric cross section may be oval shaped as shown, or may include other shapes including, for example, rectangular, diamond, elliptical, oblong, or other shapes that would occur to one skilled in the art. The implant 10 may also include a polygonal cross section such as the implant 10B shown in FIG. 6. In the embodiment shown, the implant 10B includes six sides, but other shapes, including, for example, triangular, square, or other shapes that would occur to one skilled in the art may include a different number of sides. Generally, each of these embodiments includes a substantially constant cross-section along the length of the implant 10. In other embodiments, the implant cross section may change along its length. For instance, FIG. 7 shows two dashed line representations for outer walls 20A, 20B that vary towards the ends of the implant 10. In the first instance, the cross section width of wall 20A increases suddenly from a narrower wall 20. In the second instance, the cross section width of wall 20B increases gradually from the narrower wall 20. For embodiments that include a varying cross section width, the wall thickness may vary or remain the same along the length of the implant.

Regardless of the shape of the implant 10, the tubular implant 10 may be employed alone or in combination with end caps and/or bone growth promoting materials as are known in the art. FIG. 7 also shows some possible combinations for these features. The bone growth promoting materials (BPM) 26 may include, for example, bone graft, bone morphogenetic proteins, allograft, autograft, and various types of cement, growth factors and mineralization proteins. These bone growth materials may be packed into the interior volume 24 of the non-expanding portions 14 at each end of the implant 10 to promote osseointegration of the implant to the vertebral bodies V1, V2. In a further embodiment, the bone growth promoting materials may be provided in a carrier (not shown), such as, for example, a sponge, a block, a cage, folded sheets, or paste that may be inserted into the interior volume 24.

Notably, since the interior volume 24 of the non-expanding portions 14 should not appreciably change in size or shape when the implant expands (due to exposure to elevated temperatures), the packed bone growth promoting materials should be minimally disturbed. If the volume 24 were to change size or shape, the packed growth promoting materials may loosen or dislodge from the volume 24 and reduce the effectiveness of the fusion between the implant 10 and the vertebral bodies V1, V2.

FIG. 7 further shows exemplary end plates or end caps. One exemplary end plate 28 shown at the top of FIG. 7 is sized and shaped to fit inside the end 29 of the implant 10. The end plate 28 may be pressed, bonded, or otherwise secured to the implant 10. The end plate 28 may include apertures 30 as shown in FIG. 8 that permit bony ingrowth into the interior volume 24 of the implant 10. One example of an end plate 28 suitable for the present application may be found in the Pyramesh® Titanium Mesh System available from Medtronic, Spinal and Biologics Division, in Memphis, Tenn., USA.

In another embodiment, an end cap 32 may be sized and shaped to fit around the outside of the end 33 of the implant 10. The end cap 32 may be pressed, bonded, or otherwise secured to the implant 10. The end cap 32 may include bone engaging features 34 as shown in FIG. 7 that engage the vertebral members V1, V2. In one embodiment, the end plate 28 may also include bone engaging features 34. In one embodiment, the end cap 32 may include apertures 30 to permit bony ingrowth. An end plate 28 or end cap 32 may include a curved shape to conform to the geometry of the vertebral members V1, V2. Further, the end plate 28 or end cap 32 may include an angled configuration to impart a desired angle of curvature to the spine as would be understood by those skilled in the art.

In addition, either the end plate 28 or end cap 32 may include surface features (not specifically shown) to promote bone growth and adhesion at the interface between an implant 10 and a vertebral end plate V1, V2. Examples of features used for this purpose include, for example, teeth, scales, keels, knurls, and roughened surfaces. Some of these features may be applied through post-processing techniques such as blasting, chemical etching, and coating, such as with hydroxyapatite. The bone interface surfaces, including the osteoconductive inserts, may also include growth-promoting additives such as bone morphogenetic proteins. Alternatively, pores, cavities, or other recesses into which bone may grow may be incorporated via a molding process. Other types of coatings or surface preparation may be used to improve bone growth into or through the bone-contact surfaces.

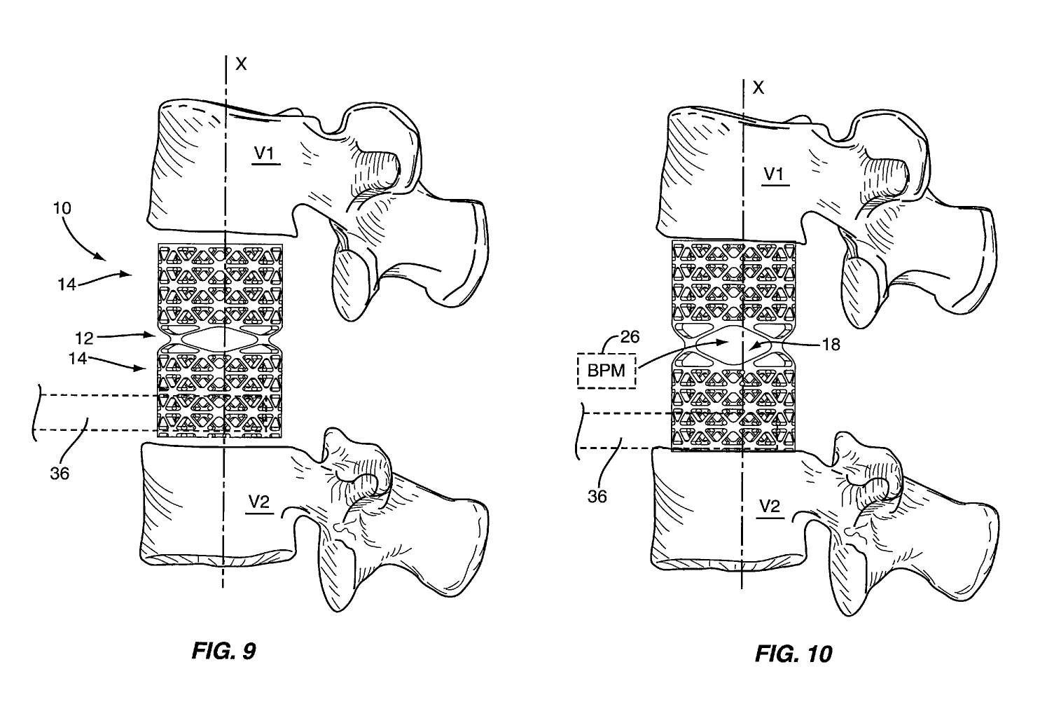

A prepared, compressed implant 10 may be inserted between vertebral members V1, V2 as shown in FIG. 9. An implant tool 36 may engage the non-expanding portion 14 of the implant 10 during insertion. The implant tool 36 may be used to accurately position the implant 10 relative to the vertebral bodies V1, V2. As suggested above, since the non-expanding portions 14 should not appreciably change in size or shape when the implant expands (due to exposure to elevated temperatures), the interface between the implant tool 36 and the implant 10 should be minimally disturbed. If the non-expanding portion 14 were to change size or shape, the positioning of the implant 10 relative to the tool 36 and the vertebral bodies V1, V2 may be adversely affected.

Once the implant 10 is positioned as desired, exposure to body temperatures (or locally applied heat) causes the expanding portion 12 to expand and fill the gap between the vertebral members V1, V2 as shown in FIG. 10. Thus, distraction of the vertebral bodies V1, V2 is achieved along longitudinal axis X, which corresponds at least generally with the longitudinal axis of the spine and of the implant 10. Once, the implant 10 has expanded as desired, the implant tool 36 may be removed. Furthermore, additional bone growth promoting materials 26 may be back-filled through the enlarged apertures 18 of the expanding portion 12 and into the interior volume 24 of the implant 10.

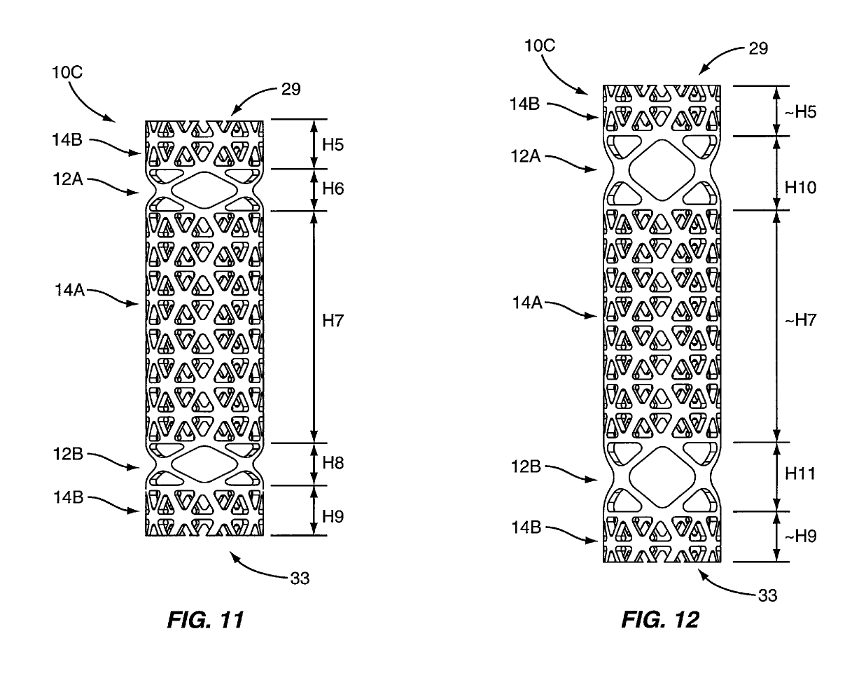

In the embodiments of the implant 10 described above, the expanding portion 12 was limited to a relatively small central portion of the implant 10. In fact, the expanding portion 12 may be located away from a central portion of the implant 10. Further, the implant may have more than one expanding portions 12A, 12B as shown in the exemplary implant 10C shown in FIGS. 11 and 12. In this embodiment, the expanding portions 12A, 12B are disposed away from a central region of the implant and towards the ends 29, 33 of the implant 10C. The extreme ends 29, 33 of the implant 10 include non-expanding portions 14B. Another non-expanding portion 14A is disposed between the expanding portions 12A, 12B. As above, the non-expanding portions 14A, 14B do not substantially change shape upon exposing the implant 10C to elevated temperatures. Thus, height dimensions H5, H7, and H9 remain substantially the same before and after expansion between the compressed state shown in FIG. 11 and the expanded state shown in FIG. 12. On the other hand, the expanding portions 12A, 12B expand from initial heights H6, H8 to expanded heights H10, H11 as shown.

In an embodiment shown in FIGS. 13 and 14, the expanding portion 12C extends over substantially all of the implant 10D except for the extreme ends 29, 33. Thus, implant 10D includes an expanding portion 12C that extends over a larger percentage of the implant 10D as compared to above-described embodiments. As a result, the implant 10D may undergo an increased change in height as the expanding portion 12C expands from height H12 to H13. Again, the non-expanding portions 14B do not substantially change shape upon exposing the implant 10D to elevated temperatures. Thus, height dimensions H5 and H9 remain substantially the same before and after expansion between the compressed state shown in FIG. 13 and the expanded state shown in FIG. 14. Notably, in the embodiment shown in FIGS. 13 and 14, the enlarged apertures 18A in the expanding region 12C include a non-geometric shape with non-linear linking segments 16A that allow the implant 10D to collapse axially in the direction of the longitudinal axis X, while minimizing radial displacement in a direction transverse to the longitudinal axis X.

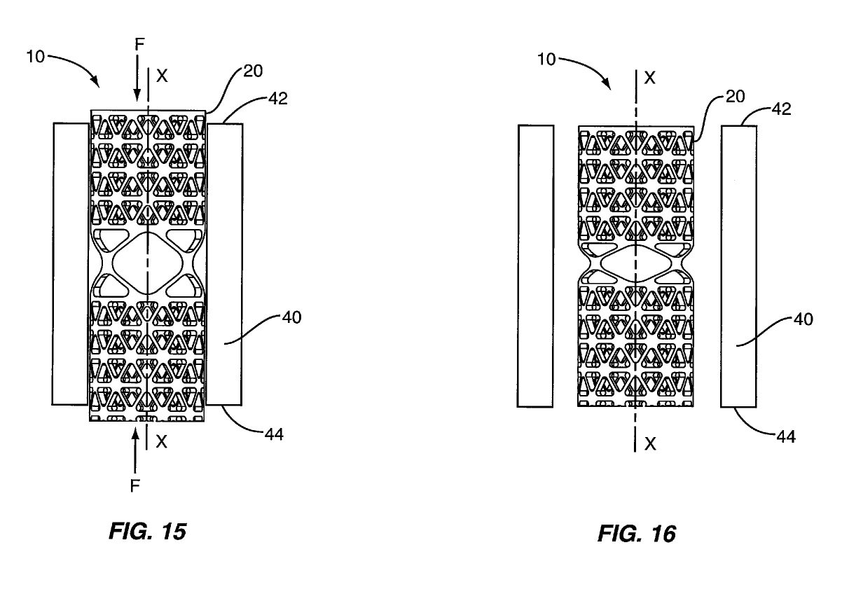

Another method of limiting radial displacement of the implants 10 is to physically constrain the implant 10 during compression while chilling the implant 10 as shown in FIGS. 15 and 16. In the technique shown, the implant 10 is chilled to a desired temperature sufficient to transform the shape-memory material to a state in which the compression sets. The implant 10 may be constrained using a mold 40. In the embodiment shown, the mold 40 may be radially adjustable to abut the outer walls 20 of the implant 20. In other embodiments, the mold 40 is preformed and includes a cylindrical shape that matches the outer width of the implant 10. Once the implant 10 is constrained with the mold 40, a compression force F is applied to compress the implant 10. A top 42 and bottom 44 surface of the mold 40 may be used as a guide to limit the amount of compression. Other measuring techniques may be used to determine an appropriate amount of compression. Then, once the compression sets, the adjustable mold 40 may be separated from the implant 10, leaving the compressed, chilled implant 10. For pre-formed molds 40, the implant 10 may be removed axially by sliding the implant 10 out of the mold 40 along the direction of the longitudinal axis X.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For instance, the implants described herein have all included a non-expanding portion disposed at the longitudinal ends of the implant. In other embodiments, the ends of the implant may include expanding portions. The present embodiments are, therefore, to be considered in all respects as illustrative and not restrictive, and all changes coming within the meaning and equivalency range of the appended claims are intended to be embraced therein.

What is claimed is:

1. A vertebral implant for insertion between vertebral bodies in a patient, the vertebral implant comprising:
a tubular member extending along a longitudinal axis and including a first region comprised of a shape-memory material, the first region including a first longitudinal height when the implant is maintained at a temperature below a threshold temperature, the first region including a second longitudinal height when the implant is maintained at a temperature at or above the threshold temperature,
the tubular member further including a second region including a third longitudinal height when the implant is maintained at a temperature below the threshold temperature, the second region remaining at substantially the same third longitudinal height when the implant is maintained at a temperature at or above the threshold temperature.

2. The vertebral implant of claim 1 wherein the first and second regions include a substantially similar width.

3. The vertebral implant of claim 1 further comprising an end member coupled to the tubular member.

4. The vertebral implant of claim 1 wherein the second region is disposed at an end of the tubular member.

5. The vertebral implant of claim 1 wherein the second region is comprised of the shape-memory material.

6. The vertebral implant of claim 1 wherein the tubular member is substantially cylindrical.

7. A vertebral implant for insertion between vertebral bodies in a patient, the vertebral implant comprising:
a tubular member extending along a longitudinal axis and including a first end region, an opposite second end region, and an intermediate region,
the intermediate region comprised of a shape-memory material, the intermediate region including a first longitudinal height when the implant is maintained at a temperature below a threshold temperature, the intermediate region including a second longitudinal height when the implant is maintained at a temperature at or above the threshold temperature,
the first end region and the second end region including a respective tubular size and shape that remains substantially the same when the implant is maintained at a temperature above or below the threshold temperature.

8. The vertebral implant of claim 7 wherein the intermediate and end regions include a substantially similar width.

9. The vertebral implant of claim 7 further comprising a first and second end piece respectively coupled to the first and second end regions.

10. The vertebral implant of claim 7 wherein the first and second end regions are comprised of the shape-memory material.

11. The vertebral implant of claim 10 wherein the shape-memory material is a polymer.

12. The vertebral implant of claim 10 wherein the shape-memory material is a metal alloy.

13. The vertebral implant of claim 7 wherein the tubular member is substantially cylindrical.

14. A method of stabilizing a spine by inserting a vertebral implant between first and second vertebral bodies in a patient, the method comprising:
maintaining the implant at a temperature below a shape transition temperature for the implant;
inserting the implant between the vertebral bodies;
exposing the implant to an elevated temperature above the shape transition temperature for the implant;
causing a first tubular portion of the implant to expand in a longitudinal direction while a second tubular portion of the implant remains at a substantially constant height; and
respectively engaging opposite ends of the implant with the first and second vertebral bodies.

15. The method of 14 further comprising compressing the implant in the longitudinal direction while maintaining the implant at the temperature below the shape transition temperature.

16. The method of claim 15 wherein the step of compressing the implant in the longitudinal direction further comprises constraining the implant to prevent radial deflection in a direction transverse to the longitudinal direction.

17. The method of claim 14 further comprising inserting bone growth promoting materials into an interior of the second tubular portion of the implant.

18. The method of claim 14 further comprising inserting bone growth promoting materials into an interior of the first tubular portion of the implant.

19. The method of claim 14 wherein second tubular portion is disposed at one of the opposite ends of the implant.

20. The method of claim 14 wherein the step of inserting the implant between the vertebral bodies further comprises grasping the second tubular portion of the implant.

21. The method of claim 14 wherein the step of exposing the implant to an elevated temperature above the shape transition temperature for the implant comprises exposing the implant to a normal body temperature.

22. A method of stabilizing a spine by inserting a vertebral implant between first and second vertebral bodies in a patient, the method comprising:
maintaining the implant at a first longitudinal height at a temperature below a shape transition temperature for the implant;
inserting bone growth promoting materials into an interior volume at opposite ends of the implant;
inserting the implant between the vertebral bodies;
exposing the implant to an elevated temperature above the shape transition temperature for the implant;
causing an intermediate portion of the implant to expand in a longitudinal direction and causing the implant to expand to a greater second height; and
respectively engaging the opposite ends of the implant with the first and second vertebral bodies.

23. The method of claim 22 wherein the step of maintaining the implant at a first longitudinal height comprises compressing the implant in the longitudinal direction while constraining the implant to prevent radial deflection in a direction transverse to the longitudinal direction.

24. The method of claim 22 further comprising inserting bone growth promoting materials into an interior of the intermediate portion of the implant.

25. The method of claim 22 wherein the step of inserting the implant between the vertebral bodies further comprises grasping one of the opposite ends of the implant.

26. The method of claim 22 wherein the opposite ends includes a respective tubular size and shape that remains substantially the same when the implant is maintained at a temperature above or below the shape transition temperature.

27. The method of claim 22 wherein the step of exposing the implant to an elevated temperature above the shape transition temperature for the implant comprises exposing the implant to a normal body temperature.
